(19) United States
(12) Patent Application Publication
(10) Pub. No.: US 2024/0109977 A1
(43) Pub. Date: Apr. 4, 2024

(54) ANTI-CD38 ANTIBODIES AND THEIR USES

(71) Applicant: MORPHOSYS AG, Planegg (DE)

(72) Inventors: Stefan Steidl, Munich (DE); Stefan Härtle, Mammendorf (DE); Rainer Boxhammer, Kolbermoor (DE)

(21) Appl. No.: 18/261,376

(22) PCT Filed: Jan. 14, 2022
(86) PCT No.: PCT/EP2022/050704

§ 371 Date: Jul. 13, 2023

(30) Foreign Application Priority Data
Jan. 14, 2021 (EP) 21151575.4

(51) Int. Cl. C07K 16/28; A61P 13/12; A61P 37/06

(52) U.S. Cl. CPC C07K 16/2896; A61P 13/12; A61P 37/06; A61K 2039/505

(57) ABSTRACT

The present invention relates to the use of an anti-CD38 antibody or antibody fragment in the prophylaxis and/or treatment of diseases directly caused by immune complex deposits. In accordance with the present invention, an anti-CD38 antibody is effective in the treatment of IgA nephropathy (IgAN).

### FIELD OF THE INVENTION

The present invention relates to an antibody or antibody fragment specific for CD38 for use in the treatment and/or prophylaxis of diseases caused by the deposition of immune complexes (ICs). In particular, the invention provides methods for the reduction of ICs by (i) depletion of immunoglobulin-secreting cells and/or (ii) depletion of antibody-secreting cells, wherein the antibody secreted by those cells is directed against the immunoglobulin, using an anti-CD38 agent. In accordance with the present invention, an anti-CD38 antibody, alone or in combination with one or more immunosuppressive drugs, can be effective in the treatment and/or prophylaxis of IgA nephropathy (IgAN) and lupus nephritis (LN). An anti-CD38 antibody for use according the present invention includes, but is not limited to MOR202 (felzartamab).

### BACKGROUND

### Immune Complex Mediated Diseases

Immune complexes (ICs) are often the pathogenic agents in certain diseases. It is well known that ICs formed in the circulation between an antigen (e.g. an antibody) and a bivalent antibody is the major pathogenic mechanism responsible for glomerular and vascular lesions, for example in glomerulonephritis and arteritis. Tissue injury by ICs is usually mediated by activation of the complement system, generation of chemotactic factors, and/or attraction of immune cells. The term ‘immune complex mediated disease’ is widely used to refer to diseases in which circulating ICs are believed to play a leading role. Although most IC-mediated diseases are initiated by deposition of complexes from the circulation, complexes formed locally within tissues can also produce injury. ICs that cause disease may be composed of antibodies bound to either self-antigens or foreign antigens. The pathologic features of IC-mediated diseases reflect the site of IC deposition and are not determined by the cellular source of the antigen. Therefore, IC-mediated diseases tend to affect multiple organs, although some are particularly susceptible, such as kidneys and joints. The term ‘immune complex mediated disease’ implies that ICs are the basic mediators of injury. However, circulating complexes may also be present in diseases in which other pathogenic mechanisms are of far greater importance, and in some instances, the complexes in fact appear to be of little if any significance. Importantly, IC-mediated injury as used herein refers to situations only in which the complexes are formed in extracellular sites, either in the circulation or locally within tissues. This definition excludes damage brought about by antibodies that combine with cell surface antigens as typically in type II immune reactions. For example, diseases resulting from autoantibodies against basement membrane (e.g., anti-glomerular basement membrane disease, anti-PLAR2 membranous glomerulonephritis) are not classified as IC-mediated diseases herein. The present disclosure relates to circulating ICs as in type III immune responses. Usually large IC aggregates, or insoluble ICs, are cleared by the mononuclear phagocyte system in liver and spleen, but small soluble ICs have a propensity for tissue deposition. IC deposition is influenced by systemic factors, physiochemical properties of the ICs, and tissue-specific hemodynamics and may be triggered by alterations in vascular permeability induced by cytokines and/or lipid mediator. Very often circulating ICs first localize within the vasculature and then translocate into extravascular tissue.

### Anti-CD20 Resistant B Cell Depletion

B cell depletion with the anti-CD20 antibody rituximab (RTX) is widely used for the treatment of autoimmune diseases including IC-mediated diseases. However, the proportion of patients achieving a long-term remission after treatment by anti-CD20 antibody varies greatly depending on the disease and clinical context and many patients frequently relapse after anti-CD20 B cell depletion therapy (Lafayette R A et al. (2017) J Am Soc Nephrol.; 28(4):1306-1313).

Three scenarios may explain these relapses: (i) a new tolerance breakdown may recruit newly formed naïve B cells into the autoimmune repertoire; (ii) autoreactive memory B cells that were resistant to RTX may be reactivated and (iii) antibody-secreting long-lived plasma cells leading to immune complexes survived in their niches.

The present disclosure provides improved options for treatment and/or prevention of IC-mediated diseases, in particular for IgA nephropathy and lupus nephritis.

### IgA Nephropathy

IgA nephropathy (IgAN), also known as Berger's disease is the most prevalent chronic glomerular disease worldwide. The disease derives its name from deposits of immunoglobulin A (IgA) in the glomerular mesangium. The subclass IgA1 is one of the two immunoglobulin types (the other is IgD) that is O-glycosylated on a number of serine and threonine residues in the proline-rich hinge region. Aberrant glycosylation of IgA1 appears to lead to polymerization of IgA1 molecules in certain tissues, especially the glomerular mesangium. The triggers and the site for production of galactose-deficient IgA1 (Gd-IgA1) are not known In IgAN. Plasma cells, including long-lived plasma cells, are likely the main source of the corresponding antibodies against Gd-IgA1. A central finding in IgAN patients is the presence of circulating and glomerular ICs comprised of Gd-IgA1 and autoantibody (mainly IgG) directed against the hinge region O-glycans, and C3. These ICs are nephrogenic and contribute to glomerular inflammation and mesangial proliferation. Ultimately, activation of the renin angiotensin system (RAS) and the complement system contributes to glomerulosclerosis and tubulo-interstitial fibrosis, leading to loss of renal function. About 25-30% of patients with IgAN progress to end-stage renal disease (ESRD) within 20-25 years of presentation (KDIGO Clinical Practice Guideline on Glomerular Diseases, 2020). Major risk factors for progression to ESRD are persistent proteinuria, hypertension, and reduced glomerular filtration rate (GFR) (Fellstroem B C et al. (2017) Lancet; 389(10084):2117-2127). Treatment of IgAN is focused on non-immunosuppressive strategies as standard of care, to slow the rate of disease progression: blood pressure control, inhibition of the RAS, and lifestyle modification (i.e. weight reduction, exercise, smoking cessation, and dietary sodium restriction etc.). Multiple studies demonstrate that sustained proteinuria is the most powerful predictor of long-term kidney outcome and that reduction in proteinuria is associated with improved kidney outcome regardless of the nature of the intervention, thereby establishing reduction in proteinuria as a valid surrogate marker of improved kidney outcome in IgAN (Thompson A et al. (2019) Clin J Am Soc Nephrol; 14: 469-481). Typical target for proteinuria reduction in these trials was <1 g/day. Thus, reduction of proteinuria to this level is a reasonable target for interventions in patients with IgAN who remain at high risk of progressive chronic kidney disease. For patients with persistent proteinuria of more than 1 g/day and GFR greater than 50 mL/min per 1.73 m² despite 6 months of optimized RAS blockade, 6 months of treatment with high-dose systemic corticosteroids is suggested. However, clinical benefit is not established and use of high-dose systemic corticosteroids is associated with increased risks of adverse events and sequelae such as serious infections, hypertension, weight gain, diabetes, and osteoporosis (Pozzi C (2016) J Nephrol. (1):21-5). Systemic corticosteroids should be given with extreme caution or avoided entirely in patients with GFR less than 30 mL/min, diabetes, obesity, latent infections (e.g. viral hepatitis, tuberculosis), secondary disease (e.g. cirrhosis), active peptic ulceration or uncontrolled psychiatric disease. Clinical trials for treatment of IgAN with azathioprine, calcineurin inhibitors and rituximab have not provided documented evidence of efficacy (Pozzi C (2016) J Nephrol. (1):21-5; Rauen T et al. (2020) Kidney Int. 98(4):1044-1052). Mycophenolate mofetil (MMF) reportedly reduced proteinuria and stabilized GFR in Chinese patients (Tang et al. (2005) Kidney Int. 68:802-812., but not in Caucasian patients (Frisch G et al (2005) Nephrol Dial Transplant; 20:2139-2145).

Although evidence for RTX efficacy in some glomerular diseases is promising, early results in IgAN are not encouraging. For example, in a randomized, controlled trial of rituximab in IgAN with proteinuria and renal dysfunction (NCT00498368) treatment with RTX failed to significantly reduce proteinuria or benefit renal function (Lafayette R A et al. (2017) J Am Soc Nephrol.; 28(4):1306-1313).

Therefore, improved treatment options with a more favorable risk-benefit profile and universal efficacy are needed for patients with IgAN.

### Lupus Nephritis

Lupus nephritis (LN) is an inflammation of the kidneys that occurs as consequence of systemic lupus erythematosus (SLE) and occurs in 20% to 60% of patients with SLE. Histopathologically, LN is a glomerulonephritis with IC deposits as result of formation of autoantibodies against nuclear antigens. Further pathological changes may include tubulointerstitial nephritis and vascular changes with IC deposits in the vessels and microangiopathy. Based on disease severity, LN can be classified in at least six categories. Class I disease (minimal mesangial glomerulonephritis) appears with normal clinical urinalysis and an unobtrusive appearance under light microscopy, but reveals mesangial deposits when analyzed by electron microscopy. Class II disease (mesangial proliferative glomerulonephritis) is characterized by mesangial hypercellularity and matrix expansion. Haematuria with or without proteinuria may be present. Class III disease (focal glomerulonephritis) is noted by sclerotic lesions involving less than 50% of the glomeruli, which can be segmental or global with endocapillary or extracapillary proliferative lesions. Clinically, haematuria, proteinuria, hypertension, and elevated serum creatinine are present. Class IV disease (diffuse proliferative nephritis) is the most severe, and the most common subtype. More than 50% of glomeruli are involved that show segmental or global, with endocapillary or extracapillary proliferative lesions. Clinically, haematuria and proteinuria are present, frequently with nephrotic syndrome, hypertension, hypocomplementemia, elevated anti-dsDNA antibody titres and elevated serum creatinine. Class V disease (membranous glomerulonephritis) is characterized by diffuse thickening of the glomerular capillary wall. Clinically, stage V presents with signs of nephrotic syndrome. Stage V can also lead to thrombotic complications such as renal vein thromboses or pulmonary emboli. Class VI, or advanced sclerosing LN is represented by global sclerosis involving more than 90% of glomeruli. This stage is characterized by slowly progressive kidney dysfunction.

Drug regimens prescribed for LN include cyclophosphamide with corticosteroids, MMF, azathioprine with corticosteroids, and tacrolimus. Cyclophosphamide-containing regimens have a high incidence of adverse events, such as serious infection, hair-loss, and infertility. Furthermore, response to treatment is often slow, and even if remission is achieved, the risk of relapse remains considerable. MMF has emerged as a less toxic alternative to cyclophosphamide and it appears that MMF and cyclophosphamide with corticosteroids are equally effective in achieving remission of the disease. Lupus nephritis (LN) is often resistant to standard of care immunosuppression and relapses are common after achieving clinical remission.

Overall, LN remains a serious condition, leading to end-stage kidney disease in 15% of patients after 10 years. Improved treatment options (e.g. steroid sparing) are highly needed for patients suffering from LN.

### SUMMARY OF THE INVENTION

The present invention provides antibodies or antibody fragments specific for CD38, for use in the treatment and/or prevention of autoimmune diseases with immune complex deposition as being the major pathological mechanistic cause of said autoimmune disease. In particular, the anti-CD38 antibody or antibody fragment is for use in the treatment and/or prevention of IgA nephropathy and/or lupus nephritis. Preferably, the anti-CD38 antibody or antibody fragment is for use in the treatment and/or prevention of IgA nephropathy.

Furthermore, the present invention provides pharmaceutical compositions comprising a therapeutically effective amount of an antibody or antibody fragment specific for CD38 for use in the treatment and/or prevention of IgA nephropathy.

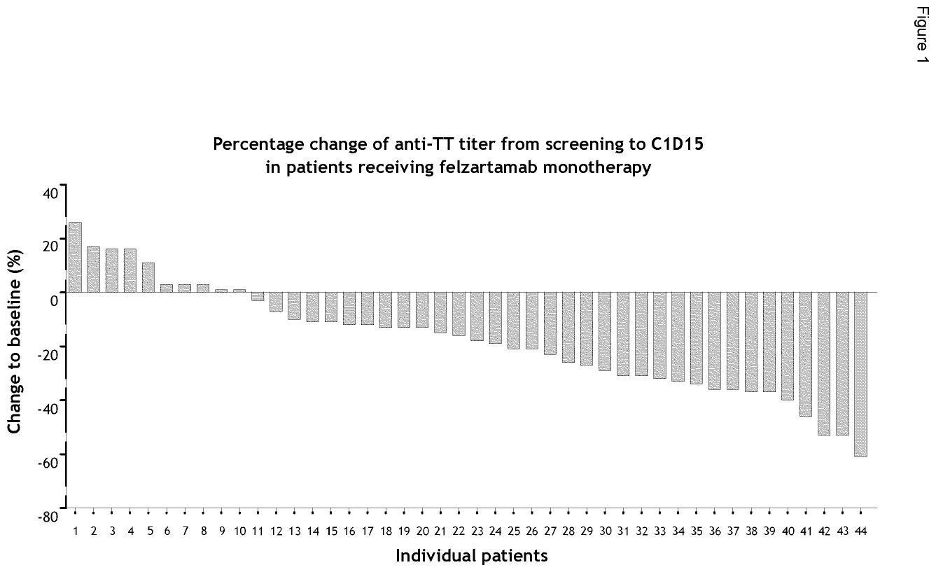

Felzartamab (MOR202) is a known monoclonal human anti-CD38 antibody that targets antibody-secreting cells such as plasmablasts and plasma cells primarily via antibody-dependent cell-mediated cytotoxicity (ADCC) and antibody-dependent cell-mediated phagocytosis (ADCP). During a clinical trial with MOR202, efficient killing of neoplastic, tumorous, malign plasma cells (i.e. multiple myeloma cells) as well as benign plasma cells has been demonstrated. In patients suffering from multiple myeloma (MM), plasma cell depletion by MOR202 leads to a significant reduction in M-Protein. In contrast to other anti-CD38 antibodies, MOR202 is expected to spare cells with low CD38 expression (e.g. NK cells) and therefore offers an optimal safety profile. The effect of MOR202 on plasma cells has been shown by assessment of the anti-Tetanus Toxoid (anti-TT) antibody titer in the serum as marker for depletion of specific plasma cells. After MOR202 administration, serum anti-TT antibody levels were significantly reduced as compared to the baseline prior to MOR202 administration (WO2020187718), showing a direct effect of MOR202 treatment on the concentration of specific antibodies (FIG. 1).

MOR202 administration also efficiently reduces circulating and deposited ICs, primarily in the glomeruli of the kidneys in IgAN patients, resulting in an improvement of kidney function and well-being of these patients.

### DETAILED DESCRIPTION OF THE INVENTION

FIG. 1

Proof-of-mechanism is supported by a reduction in anti-tetanus toxoid titers, a surrogate marker for effect on plasma cells.

### DEFINITIONS

The term “CD38” refers to a protein known as CD38, having the following synonyms: ADP-ribosyl cyclase 1, cADPr hydrolase 1, Cyclic ADP-ribose hydrolase 1, T10. Human CD38 (UniProt P28907) has the following amino acid sequence:

(SEQ ID NO.: 9)
MANCEFSPVSGDKPCCRLSRRAQLCLGVSILVLILVVVLAVVVPRWRQQ
WSGPGTTKRFPETVLARCVKYTEIHPEMRHVDCQSVWDAFKGAFISKHP
CNITEEDYQPLMKLGTQTVPCNKILLWSRIKDLAHQFTQVQRDMFTLED
TLLGYLADDLTWCGEFNTSKINYQSCPDWRKDCSNNPVSVFWKTVSRRF
AEAACDVVHVMLNGSRSKIFDKNSTFGSVEVHNLQPEKVQTLEAWVIHG
GREDSRDLCQDPTIKELESIISKRNIQFSCKNIYRPDKFLQCVKNPEDS
SCTSEI

CD38 is a type II transmembrane glycoprotein and an example of an antigen that is highly expressed on antibody-secreting cells (including autoantibody-secreting plasmablasts and plasma cells). Functions ascribed to CD38 include both receptor-mediated adhesion and signaling events and (ecto-) enzymatic activity. As an ectoenzyme, CD38 uses NAD+ as substrate for the formation of cyclic ADP-ribose (cADPR) and ADPR, but also of nicotinamide and nicotinic acid-adenine dinucleotide phosphate (NAADP). cADPR and NAADP have been shown to act as second messengers for Ca2+ mobilization. By converting NAD+ to cADPR, CD38 regulates the extracellular NAD+ concentration and hence cell survival by modulation of NAD-induced cell death (NCID). In addition to signaling via Ca2+, CD38 signaling occurs via cross-talk with antigen-receptor complexes on T and B cells or other types of receptor complexes, e.g. MHC molecules, and is in this way involved in several cellular responses, but also in switching and secretion of IgG antibodies.

The term “anti-CD38 antibody”, as used herein, includes anti-CD38 binding molecules in its broadest sense; any molecule which specifically binds to CD38 or inhibits the activity or function of CD38, or which by any other way exerts a therapeutic effect on CD38 is included. Any molecule that interferes or inhibits CD38 functionality is included. The term “anti-CD38 antibody” includes, but is not limited to, antibodies specifically binding to CD38, alternative protein scaffolds binding to CD38, nucleic acids (including aptamers) specific for CD38 or small organic molecules specific for CD38.

Antibodies specific for CD38 are described for example in WO199962526 (Mayo Foundation); WO200206347 (Crucell Holland); US2002164788 (Jonathan Ellis); WO2005103083, WO2006125640, WO2007042309 (MorphoSys), WO2006099875 (Genmab), and WO2008047242 (Sanofi-Aventis). Combinations of antibodies specific for CD38 and other agents are described for example in WO200040265 (Research Development Foundation); WO2006099875 and WO2008037257 (Genmab); and WO2010061360, WO2010061359, WO2010061358 and WO2010061357 (Sanofi Aventis). CD38-targeting antibodies are broadly used in multiple myeloma (reviewed in Frerichs K A et al. 2018, Expert Rev Clin Immunol; 14(3):197-206). Further uses of anti-CD38 antibodies are described for example in WO2015130732, WO2016089960, WO2016210223 (Janssen), WO2018002181 (UMC Utrecht), WO2019020643 (ENCEFA) and WO2020187718 (MorphoSys) which are all incorporated by reference in their entireties.

Preferably, an anti-CD38 antibody for the use as described herein is an antibody specific for CD38. More preferably, an anti-CD38 antibody is an antibody or antibody fragment, such as a monoclonal antibody, specifically binding to CD38 and deleting specific CD38 positive B cells, plasma cells, plasmablasts and any other CD38 positive antibody-secreting cells. Such an antibody may be of any type, such as a murine, a rat, a chimeric, a humanized or a human antibody.

A “human antibody” or “human antibody fragment”, as used herein, is an antibody or antibody fragment having variable regions in which the framework and CDR regions are from sequences of human origin. If the antibody contains a constant region, the constant region also is from such sequences. Human origin includes, but is not limited to human germline sequences, or mutated versions of human germline sequences or antibody containing consensus framework sequences derived from human framework sequences analysis, for example, as described in Knappik et al., (2000) J Mol Biol 296:57-86). Human antibodies can be isolated e.g. from synthetic libraries or from transgenic mice (e.g. Xenomouse). An antibody or antibody fragment is human if its sequence is human, irrespective of the species from which the antibody is physically derived, isolated, or manufactured.

The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g. Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948.

A “humanized antibody” or “humanized antibody fragment” is defined herein as an antibody molecule, which has constant antibody regions derived from sequences of human origin and the variable antibody regions or parts thereof or only the CDRs are derived from another species. For example, a humanized antibody can be CDR-grafted, wherein the CDRs of the variable domain are from a non-human origin, while one or more frameworks of the variable domain are of human origin and the constant domain (if any) is of human origin.

The term “chimeric antibody” or “chimeric antibody fragment” is defined herein as an antibody molecule, which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

The term “isolated antibody” refers to an antibody or antibody fragment that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies, which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “monoclonal antibody” as used herein refers to a preparation of antibody molecules of single molecular composition. A monoclonal antibody composition displays a unique binding site having a unique binding specificity and affinity for particular epitopes.

In addition, as used herein, an “immunoglobulin” (Ig) hereby is defined as a protein belonging to the class IgG, IgM, IgE, IgA, or IgD (or any subclass thereof), and includes all conventionally known antibodies and functional fragments thereof.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment, which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as “single chain Fragment (scFv)”). Such single chain antibodies are to be encompassed within the term “antibody fragment”. Antibody fragments can also be incorporated into single domain antibodies, maxibodies, minibodies, intrabodies, diabodies, triabodies, tetrabodies, v-NAR and bis-scFv. Antibody fragments can be grafted into scaffolds based on polypeptides such as Fibronectin type III (Fn3). Antibody fragments can be incorporated into single chain molecules comprising a pair of tandem Fv segments (VH-CH1-VH-CH1) which, together with complementary light chain polypeptides, form a pair of antigen-binding sites). The present disclosure provides therapeutic methods comprising the administration of a therapeutically effective amount of an anti-CD38 antibody as disclosed to a subject in need of such treatment. A “therapeutically effective amount” or “effective amount”, as used herein, refers to the amount of an antibody specific for CD38, necessary to elicit the desired biological response. In accordance with the present disclosure, the therapeutic effective amount is the amount of an antibody specific for CD38 necessary to treat and/or prevent immune complex mediated diseases and symptoms associated with said diseases. An effective amount for a particular individual may vary, depending on factors such as the condition being treated, the overall health of the patient, the method route and dose of administration and the severity of side effects (Maynard, et al. (1996) A Handbook of SOPs for Good Clinical Practice, Interpharm Press, Boca Raton, Fla.; Dent (2001) Good Laboratory and Good Clinical Practice, London, UK).

As used herein, the terms “treat”, “treating”, or the like, mean to alleviate symptoms, eliminate the causation of symptoms either on a temporary or permanent basis, or to prevent or slow the appearance of symptoms of the named disorder or condition.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset. “Prevention” refers to methods which aim to prevent the onset of a disease or its symptoms or which delay the onset of a disease or its symptoms.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

By co-administration is included any means of delivering two or more therapeutic agents to the patient as part of the same treatment regimen, as will be apparent to the skilled person. Whilst the two or more agents may be administered simultaneously in a single formulation, i.e. as a single pharmaceutical composition, this is not essential. The agents may be administered in different formulations and at different times. The therapies (e.g., prophylactic or therapeutic agents) of the combination therapies of the present disclosure can be administered concomitantly or sequentially to a subject. The therapy (e.g., prophylactic or therapeutic agents) of the combination therapies of the present disclosure can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising antibodies or antibody fragments of the disclosure are administered to a subject in a sequence and within a time interval such that the antibodies of the disclosure can act together with the other therapy(ies) to provide an increased benefit than if they were administered otherwise.

“Subject” or “species”, as used herein refers to any mammal, including rodents, such as mouse or rat, and primates, such as cynomolgus monkey (Macaca fascicularis), rhesus monkey (Macaca mulatta) or humans (Homo sapiens). Preferably, the subject is a primate, most preferably a human.

As used herein, the term “a subject in need thereof” or the like, mean a human or a non-human animal patient that exhibits one or more symptoms or indicia of immune complex-mediated disease, and/or who has been diagnosed with an immune complex mediated disease (e.g. IgA nephropathy). Preferably, the subject is a primate, most preferably a human patient who has been diagnosed with IgA nephropathy or lupus nephritis.

As used herein, the term “immune complex-mediated diseases” refers to a group of diseases that are characterized by a deposit of immune complexes. For example, IC-mediated diseases may comprise Type III (Immune Complex) Hypersensitivity reactions in which IgG or IgM antibodies develop against circulating antigens and which often affect one or more tissues like skin, joints, vessels, or glomeruli of the kidney. Examples of immune complex (IC)-mediated diseases are shown in Table 1, but are not limited to the listed diseases.

TABLE 1
Examples of immune complex(IC)-mediated diseases

| | Immuncomplex | | |
|---|---|---|---|
| IC mediated | formed by | | Main target |
| Disease | antibody(ies) | antigen(s) | organ(s) |
| IgA nephropathy | anti-galactose- | galactose-deficient | kidney |
| (IgAN) | deficient | IgA1 | |
| | IgA1 antibody | antibody | |
| | (anti-GD-IgA1) | (Gd-IgA1) | |
| Lupus nephritis | antinuclear | DNA | kidney |
| | antibodies | nuclear | |
| | (ANAs) | components | |
| | anti-DNA | | |
| | anti-Ro/SS-A | | |
| | and anti- | | |
| | La/SS-B | | |
| | antibodies | | |
| Drug-induced | antinuclear | nuclear | kidney |
| immune complex- | antibodies | components | |
| mediated diffuse | and anti-Ro/SS-A | Ro/SS-A | |
| proliferative | and anti-La/SS-B | La/SSB | |
| glomerulonephritis | antibodies | DNA | |
| Henoch-Schönlein | anti-glycan | | kidney |
| purpura | antibodies | | |
| nephritis | | | |
| Vasculitis | ANCAs | Neutrophil granule | several, e.g. |
| | (anti-neutrophil | proteins, | kidney, |
| | cytoplasmic | presumably | lung, skin |
| | autoantibodies) | released from | if only the |
| | | activated | kidney is |
| | | neutrophils | affected it |
| | | myeloperoxidase | is called |
| | | serine | “renal-limited |
| | | proteinase 3 | ANCA |
| | | | vasculitis” |
| Poststreptococcal | Anti-DNase | | kidney |
| glomerulonephritis | B antibody | | |
| (PSGN) | Anti- | | |
| | streptolysin-O | | |
| | ab | | |

As used herein, the term “about” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

“Pharmaceutically acceptable” means approved or approvable by a regulatory agency of the Federal or a state government or the corresponding agency in countries other than the United States, or that is listed in the US Pharmacopoeia or other generally recognized pharmacopoeia for use in animals, and more particularly, in humans.

“Pharmaceutically acceptable vehicle” refers to a diluent, adjuvant, excipient or carrier with which an antibody or antibody fragment is administered.

Throughout this specification, unless the context requires otherwise, the words “comprise”, “have” and “include” and their respective variations such as “comprises”, “comprising”, “has”, “having”, “includes” and “including” will be understood to imply the inclusion of a stated element or integer or group of elements or integers but not the exclusion of any other element or integer or group of elements or integers.

“MOR202” is an anti-CD38 antibody, also known as “felzartamab”, “MOR03087” or “MOR3087”. The terms are used interchangeable in the present disclosure. MOR202 has an IgG1 Fc region.

The amino acid sequence of the MOR202 HCDR1 according to Kabat is: SYYMN (SEQ ID NO: 1)

(SEQ ID NO: 1)
SYYMN

The amino acid sequence of the MOR202 HCDR2 according to Kabat is: GISGDPSNTYYADSVKG (SEQ ID NO: 2)

(SEQ ID NO: 2)
GISGDPSNTYYADSVKG

The amino acid sequence of the MOR202 HCDR3 according to Kabat is: DLPLVYTGFAY (SEQ ID NO: 3)

(SEQ ID NO: 3)
DLPLVYTGFAY

The amino acid sequence of the MOR202 LCDR1 according to Kabat is: SGDNLRHYYVY (SEQ ID NO: 4)

(SEQ ID NO: 4)
SGDNLRHYYVY

The amino acid sequence of the MOR202 LCDR2 according to Kabat is: GDSKRPS (SEQ ID NO: 5)

(SEQ ID NO: 5)
GDSKRPS

The amino acid sequence of the MOR202 LCDR3 is:

(SEQ ID NO: 6)
QTYTGGASL

The amino acid sequence of the MOR202 Variable Heavy Domain is:

(SEQ ID NO: 7)
QVQLVESGGGLVQPGGSLRLSCAASGFTFSSYYMNWVRQAPGKGLEWVS
GISGDPSNTYYADSVKGRFTISRDNSKNTLYLQMNSLRAEDTAVYYCAR
DLPLVYTGFAYWGQGTLVTVSS

The amino acid sequence of the MOR202 Variable Light Domain is:

(SEQ ID NO: 8)
DIELTQPPSVSVAPGQTARISCSGDNLRHYYVYWYQQKPGQAPVLVIYG
DSKRPSGIPERFSGSNSGNTATLTISGTQAEDEADYYCQTYTGGASLVF
GGGTKLTVLGQ

The DNA sequence encoding the MOR202 Variable Heavy Domain is:

(SEQ ID NO: 10)
CAGGTGCAATTGGTGGAAAGCGGCGGCGGCCTGGTGCAACCGGGCGGCA
GCCTGCGTCTGAGCTGCGCGGCCTCCGGATTTACCTTTTCTTCTTATTA
TATGAATTGGGTGCGCCAAGCCCCTGGGAAGGGTCTCGAGTGGGTGAGC
GGTATCTCTGGTGATCCTAGCAATACCTATTATGCGGATAGCGTGAAAG
GCCGTTTTACCATTTCACGTGATAATTCGAAAAACACCCTGTATCTGCA
AATGAACAGCCTGCGTGCGGAAGATACGGCCGTGTATTATTGCGCGCGT
GATCTTCCTCTTGTTTATACTGGTTTTGCTTATTGGGGCCAAGGCACCC
TGGTGACGGTTAGCTCA.

The DNA sequence encoding the MOR202 Variable Light Domain is:

(SEQ ID NO: 11)
GATATCGAACTGACCCAGCCGCCTTCAGTGAGCGTTGCACCAGGTCAGA
CCGCGCGTATCTCGTGTAGCGGCGATAATCTTCGTCATTATTATGTTTA
TTGGTACCAGCAGAAACCCGGGCAGGCGCCAGTTCTTGTGATTTATGGT
GATTCTAAGCGTCCCTCAGGCATCCCGGAACGCTTTAGCGGATCCAACA
GCGGCAACACCGCGACCCTGACCATTAGCGGCACTCAGGCGGAAGACGA
AGCGGATTATTATTGCCAGACTTATACTGGTGGTGCTTCTCTTGTGTTT
GGCGGCGGCACGAAGTTAACCGTTCTTGGCCAG.

### Embodiments

### Antibody

In certain embodiments of the present disclosure, the antibody or antibody fragment specific for CD38 for the use according to the present disclosure comprises a variable heavy chain variable region, a variable light chain region, heavy chain, light chain and/or CDRs comprising any of the amino acid sequences of the CD38 specific antibodies as set forth in WO2007042309.

In an embodiment, said antibody or antibody fragment specific for CD38 for the use according to the present disclosure comprises a HCDR1 region comprising the amino acid sequence of SEQ ID NO: 1, a HCDR2 region comprising the amino acid sequence of SEQ ID NO: 2, a HCDR3 region comprising the amino acid sequence of SEQ ID NO: 3, a LCDR1 region comprising the amino acid sequence of SEQ ID NO: 4, a LCDR2 region comprising the amino acid sequence of SEQ ID NO: 5 and a LCDR3 region comprising the amino acid sequence of SEQ ID NO: 6.

In one embodiment, said antibody or antibody fragment specific for CD38 for the use according to the present disclosure, comprises the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6.

In an embodiment, said antibody or antibody fragment specific for CD38 for the use according to the present disclosure comprises a variable heavy chain region of SEQ ID NO: 7 and a variable light chain region of SEQ ID NO: 8.

In another embodiment the anti-CD38 antibody or antibody fragment for the use according to the present disclosure comprises a variable heavy chain region of SEQ ID NO: 7 and a variable light chain region of SEQ ID NO: 8 or a variable heavy chain region and a variable light chain region that has at least 60%, at least 70%, at least 80%, at least 90% or at least 95% identity to the a variable heavy chain region of SEQ ID NO: 7 and to the variable light chain region of SEQ ID NO: 8.

An exemplary antibody or antibody fragment for the use according to the present disclosure comprising the variable heavy chain region comprising the amino acid sequence of SEQ ID NO: 7 and a variable light chain region comprising the amino acid sequence of SEQ ID NO: 8 is the human anti-CD38 antibody known as MOR202 (felzartamab).

In one embodiment, the present disclosure refers to a nucleic acid composition comprising a nucleic acid sequence or a plurality of nucleic acid sequences encoding said antibody or antibody fragment specific for CD38 for the use according to the present disclosure, wherein said antibody or antibody fragment comprises the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6.

In another embodiment, the disclosure refers to a nucleic acid encoding an isolated monoclonal antibody or fragment thereof for the use according to the present disclosure wherein the nucleic acid comprises a VH of SEQ ID NO: 10 and a VL of SEQ ID NO: 11.

In one embodiment, the disclosed antibody or antibody fragment specific for CD38 for the use according to the present disclosure is a monoclonal antibody or antibody fragment.

In one embodiment, the disclosed antibody or antibody fragment specific for CD38 for the use according to the present disclosure is a human, humanized or chimeric antibody.

In certain embodiments, said antibody or antibody fragment specific for CD38 for the use according to the present disclosure is an isolated antibody or antibody fragment.

In another embodiment, said antibody or antibody fragment for the use according to the present disclosure is a recombinant antibody or antibody fragment.

In a further embodiment, said antibody or antibody fragment for the use according to the present disclosure is a recombinant human antibody or antibody fragment.

In a further embodiment, said recombinant human antibody or antibody fragment for the use according to the present disclosure is an isolated recombinant human antibody or antibody fragment.

In a further embodiment, said recombinant human antibody or antibody fragment or isolated recombinant human antibody or antibody fragment for the use according to the present disclosure is monoclonal.

In one embodiment, the disclosed antibody or antibody fragment for the use according to the present disclosure is of the IgG isotype. In a particular embodiment, said antibody is an IgG1.

In particular aspects of the present invention, the anti-CD38 antibody for the use according to the present disclosure is MOR202 (felzartamab).

In an embodiment, the present disclosure refers to a pharmaceutical composition comprising felzartamab (MOR202) or fragment thereof specific for CD38 and a pharmaceutically acceptable carrier or excipient for the use according to the present disclosure.

In certain embodiments, said antibody or antibody fragment specific for CD38 is an isolated monoclonal antibody or antibody fragment that specifically binds to human CD38.

### Pharmaceutical Compositions

When employed as a pharmaceutical the antibody, or antibody fragment, specific for CD38 is typically administered in a pharmaceutical composition. The compositions of the present disclosure are preferably pharmaceutical compositions comprising felzartamab (MOR202) and a pharmaceutically acceptable carrier, diluent or excipient, for the use in the treatment of IgA nephropathy (IgAN) and/or lupus nephritis (LN).

The pharmaceutically acceptable carrier should be suitable for intravenous, intramuscular, subcutaneous, parenteral, spinal or epidermal administration (e.g., by injection or infusion).

Pharmaceutically carriers enhance or stabilize the composition, or facilitate the preparation of the composition. Pharmaceutically acceptable carriers include solvents, dispersion media, coatings, antibacterial and antifungal agents, isotonic and absorption delaying agents, and the like that are physiologically compatible. In many cases, it is preferable to include isotonic agents, for example, sugars, polyalcohols such as mannitol or sorbitol, and sodium chloride in the composition.

A pharmaceutical composition of the present disclosure can be administered by a variety of routes known in the art. Selected routes of administration for antibodies or antibody fragments of the disclosure include intravenous, intramuscular, intradermal, intraperitoneal, subcutaneous, spinal or other parenteral routes of administration, for example by injection or infusion.

The antibody, or antibody fragment, specific for CD38 is preferably formulated as injectable composition. In preferred aspects, the anti-CD38 antibody of the present disclosure is administered intravenously. In other aspects, the anti-CD38 antibody of the present disclosure is administered, subcutaneously, intraarticularly or intra-spinally.

An important aspect of the present disclosure is a pharmaceutical composition that is able to mediate killing of CD38-expressing antibody-secreting cells (e.g. plasmablasts, plasma cells) by ADCC and ADCP.

### Methods of Treatment

In one embodiment, the present disclosure provides an anti-CD38 antibody or antibody fragment, or a pharmaceutical composition comprising an anti-CD38 antibody or antibody fragment, for use in the treatment of immune complex-mediated disease in a subject.

In one embodiment, an anti-CD38 antibody or antibody fragment, or a pharmaceutical composition comprising an anti-CD38 antibody or antibody fragment, for use in the treatment of an immune complex—mediated kidney disease is provided.

In one embodiment, the immune complex—mediated disease is selected from IgA nephropathy, lupus nephritis, Henoch-Schönlein purpura nephritis, post-streptococcal glomerulonephritis or Drug-induced immune complex-mediated diffuse proliferative glomerulonephritis.

In a particular embodiment, the present disclosure provides an anti-CD38 antibody or antibody fragment, or a pharmaceutical composition comprising an anti-CD38 antibody or antibody fragment, for use in the prophylaxis and/or treatment of IgA nephropathy (IgAN) and or lupus nephritis (LN).

In another embodiment, the present disclosure provides an anti-CD38 antibody or antibody fragment, or a pharmaceutical composition comprising an anti-CD38 antibody or antibody fragment, for use in the prophylaxis and/or treatment of galactose-deficient IgA1 antibody (Gd-IgA1) and anti-galactose-deficient IgA1 antibody (anti-GD-IgA1) positive IgA nephropathy.

In a particular embodiment, the present disclosure provides an anti-CD38 antibody or antibody fragment comprising the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6 for use in the prophylaxis and/or treatment of IgA nephropathy (IgAN) and/or lupus nephritis (LN).

In another aspect, the present disclosure provides an anti-CD38 antibody or antibody fragment comprising a variable heavy chain region of SEQ ID NO: 7 and a variable light chain region of SEQ ID NO: 8 for use in the prophylaxis and/or treatment of IgA nephropathy (IgAN) and/or lupus nephritis (LN).

In a particular aspect, the present disclosure provides MOR202 (felzartamab) for use in the prophylaxis and/or treatment of IgA nephropathy (IgAN) and/or lupus nephritis (LN).

In one embodiment, the present disclosure provides an anti-CD38 antibody or antibody fragment for use in depleting CD38 expressing antibody secreting cells (preferably plasma cells), in subjects with IgA nephropathy (IgAN) and/or lupus nephritis (LN).

In a preferred embodiment, the disclosure provides an anti-CD38 antibody (e.g. MOR202) for use in reducing circulating immune complexes and/or immune complex deposits in subjects with IgAN.

In a particular embodiment, the disclosure provides an anti-CD38 antibody (e.g. MOR202) for use in reducing serum Gd-IgA1 and anti-GD-IgA1 antibody (i.e. ab titers) and/or immune complex levels in subjects with IgAN. In another aspect, the disclosure provides an anti-CD38 antibody (e.g. MOR202) for use in reducing Gd-IgA1 and anti-GD-IgA1 immune complexes deposited in kidneys of subjects with IgAN.

In a further aspect, the disclosure provides a therapeutic agent comprising an anti-CD38 antibody (e.g. MOR 202) as an active ingredient for use in reducing proteinuria in subjects with IgAN.

In some aspects, said proteinuria is a proteinuria of up to 6.0 g/day, preferably up to 5.0 g/day, more preferably up to 4.0 g/day (total protein based on 24-hour urine collection). In some aspects, the subject with IgAN has persistent proteinuria. In some embodiments, said persistent proteinuria is a persistent proteinuria with UPCR>1 mg/mg based on 24-hour urine collection or said persistent proteinuria is a persistent proteinuria with UPCR>0.75 mg/mg based on 24-hour urine collection, wherein at least once within 12 months prior to the administration or said use of said anti-CD38 antibody (e.g. MOR 202) said subject with IgAN has been determined to have a UPCR>1 mg/mg based on 24-hour urine collection. In some embodiments, said proteinuria is characterized by a urine-protein creatinine ratio (UPCR), based on 24-hour urine collection, of at least 0.75 mg/mg, preferably at least 1.0 mg/mg, more preferably at least 1.5 mg/mg, more preferably at least 2.0 mg/mg. In some embodiments, said proteinuria is characterized by a 24-hour urine-protein creatinine ratio (UPCR), based on 24-hour urine collection, of up to 6.0 mg/mg, preferably up to 5.0 mg/mg, more preferably up to 4.0 mg/mg.

Preferably, proteinuria is reduced below 1 g/day after administration or said use of said anti-CD38 antibody.

In another aspect, the disclosure provides a preventive and/or therapeutic agent comprising an anti-CD38 antibody (e.g. MOR202) for use in restoring, ameliorating or normalizing kidney function indicated by glomerular filtration rate (eGFR) based on the CKD-epi equation in subjects with IgA nephropathy (IgAN).

In a further aspect, the disclosure provides an anti-CD38 antibody (e.g. MOR 202) for use in the treatment of IgAN and/or LN, whereby the anti-CD38 antibody (e.g. MOR202) will be dosed depending on patient body weight in at least 2 doses, at least 5 doses, or at least 9 doses.

In another aspect, the disclosure provides an anti-CD38 antibody (e.g. MOR 202) for use in the treatment of IgAN and/or LN, whereby the anti-CD38 antibody (e.g. MOR202) will be dosed depending on patient body weight in 2 doses, in 5 doses, or in 9 doses.

In another aspect, the present disclosure provides the use of an anti-CD38 antibody or antibody fragment in the preparation of a medicament for the treatment and/or prophylaxis of immune complex-mediated disease, preferably IgA nephropathy (IgAN) and/or lupus nephritis (LN), more preferably Gd-IgA1 and anti-GD-IgA1 positive IgAN.

In other aspects, the present disclosure provides the use of an anti-CD38 antibody or antibody fragment comprising the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6 in the preparation of a medicament for the treatment and/or prophylaxis of IgA nephropathy (IgAN) and/or lupus nephritis (LN).

In other aspects, the present disclosure provides the use of an anti-CD38 antibody or antibody fragment comprising a variable heavy chain region of SEQ ID NO: 7 and a variable light chain region of SEQ ID NO: 8 in the preparation of a medicament for the treatment and/or prophylaxis of IgA nephropathy (IgAN) and/or lupus nephritis (LN).

In a further aspect, the present disclosure provides the use of MOR202 (felzartamab) in the preparation of a medicament for the treatment and/or prophylaxis of IgA nephropathy (IgAN) and/or lupus nephritis (LN).

In other aspects, the present disclosure provides the use of MOR202 (felzartamab) in the preparation of a medicament for the treatment and/or prophylaxis of Gd-IgA1 and anti-GD-IgA1 positive IgA nephropathy (IgAN).

In other aspects, the present disclosure provides the use of MOR202 (felzartamab) or pharmaceutical compositions comprising MOR202 (felzartamab), in combination with another therapeutic agent, in the preparation of a medicament for the treatment and/or prophylaxis of IgA nephropathy (IgAN) and or lupus nephritis (LN).

In one aspect, the present disclosure provides a method for the treatment and/or prophylaxis of immune complex-mediated disease, comprising administering to said subject an anti-CD38 antibody. In a particular embodiment, the immune complex-mediated disease is IgA nephropathy.

In a further aspect, the present disclosure provides a method for the treatment and or/prophylaxis of Gd-IgA1 and anti-GD-IgA1 positive IgA nephropathy (IgAN) in a subject, said method comprising administering an anti-CD38 antibody to said subject.

In some embodiments, the present disclosure provides methods of prophylaxis and/or treatment of subjects suffering from Gd-IgA1 and anti-GD-IgA1 positive IgA nephropathy, wherein said subject is resistant to treatment by other immunosuppressant therapies, comprising corticosteroids or calcineurin inhibitors or B cell depleting therapies (e.g. with Rituximab or any other anti-CD20 antibody, or anti-BAFF antibody), which methods comprise the administration of an effective amount of an anti-CD38 antibody or antibody fragment.

In one aspect, the disclosure provides methods of using an anti-CD38 antibody or antibody fragment to achieve a prophylactic or therapeutic benefit in patients with IgA nephropathy, in particular Gd-IgA1 and anti-GD-IgA1 positive IgA nephropathy.

In another aspect, the disclosure provides a method using an anti-CD38 antibody to treat and/or prevent symptoms mediated with IgA nephropathy, in particular Gd-IgA1 and anti-GD-IgA1 positive IgA nephropathy.

In another aspect, the disclosure provides a method for reducing the incidence of immune-complex-deposit-mediated disease symptoms, ameliorating immune-complex-deposit-mediated disease symptoms, suppressing immune-complex-deposit-mediated disease symptoms, palliating immune-complex-deposit-mediated disease symptoms, and/or delaying the onset, development, or progression of immune-complex-deposit mediated disease in a subject, said method comprising administering an effective amount of an anti-CD38 antibody to the subject. Particularly, the immune complex-mediated disease is IgA nephropathy

In preferred embodiments, the disclosure provides methods for treating patients with elevated levels of one or more immune-complex-deposits associated with the immune-complex-deposit mediated disease.

In other aspects, the present disclosure provides a method for the treatment and/or prevention of a disease caused by the presence of Gd-IgA1 and anti-GD-IgA1 immune complexes. In yet other aspects, the present disclosure provides a method for the treatment and/or prevention of a disease associated with the presence of Gd-IgA1 and anti-GD-IgA1 immune complex deposits.

In further aspects, the present disclosure provides a method for the treatment and/or prevention of a disease associated with the presence of DNA and/or nuclear components and anti-DNA antibody and/or anti-nuclear antibody (ANA) immune complex deposits.

In other embodiments, the disclosure provides methods to reduce immune complexes in the serum and/or tissue of subjects suffering from immune complex-mediated disease, which methods comprise the administration of an effective amount of an anti-CD38 antibody or antibody fragment.

In a preferred embodiment, the disclosure provides methods to reduce immune complexes in serum of subjects suffering from IgA nephropathy and/or lupus nephritis, which methods comprise the administration of an effective amount of an anti-CD38 antibody or antibody fragment, or one or more of the pharmaceutical compositions herein described. For example, the methods provided herein comprise administering an anti-CD38 antibody to patients with elevated levels of Gd-IgA1 and anti-GD-IgA1 antibodies and immune complexes thereof. In other aspects, the methods provided herein comprise administering an anti-CD38 antibody to patients with elevated levels of anti-nuclear antibodies (ANAs) and immune complexes thereof

In one embodiment, the reduction (change) of immune complexes in serum of subjects suffering from IgA nephropathy and/or lupus nephritis is at least 5%, at least 10%, at least 15%, at least 20%, at least 25%, at least 30%, at least 35%, at least 40%, at least 45%, or at least 50% compared to baseline after administering an anti-CD38 antibody or antibody fragment, or one or more of the pharmaceutical compositions herein described.

In another embodiment, the disclosure provides methods for treating and/or prophylaxis of proteinuria associated with IgA nephropathy and/or lupus nephritis in a subject, which methods comprise the administration of an effective amount of an anti-CD38 antibody or antibody fragment, or one or more of the pharmaceutical compositions herein described.

In another aspect, the disclosure provides methods for preventing the decline of renal function in an individual with IgA nephropathy and/or lupus nephritis, which methods comprise the administration of an effective amount of an anti-CD38 antibody, or antibody fragment, or one or more of the pharmaceutical compositions herein described.

In further embodiments, the present disclosure refers to a method for the treatment of IgA nephropathy and/or lupus nephritis in a subject, comprising administering to the subject a pharmaceutical composition comprising an anti-CD38 antibody or antibody fragment that binds to a CD38 expressing cell and leads to the depletion of such CD38 expressing cell.

In a preferred embodiment, the present disclosure refers to a method for the treatment of IgA nephropathy and/or lupus nephritis in a subject, comprising administering to the subject a pharmaceutical composition comprising an anti-CD38 antibody or antibody fragment that binds to a CD38 expressing antibody-secreting cell and leads to the depletion of such CD38 expressing antibody-secreting cell, while sparing other (antibody-non-secreting) cells with low CD38 expression such as NK cells or the like.

In a particular preferred embodiment, the present disclosure refers to a method for the treatment of IgA nephropathy and/or lupus nephritis in a subject, comprising administering to the subject a pharmaceutical composition comprising an anti-CD38 antibody or antibody fragment that binds to a CD38 expressing antibody-secreting cell and leads to the depletion of such CD38 expressing antibody-secreting cell, wherein the antibody shows a significant higher specific cell killing on antibody-secreting cells than on NK cells.

In one embodiment, the present disclosure refers to a method for the treatment of IgA nephropathy and/or lupus nephritis in a subject, comprising administering to the subject a pharmaceutical composition comprising an anti-CD38 antibody or antibody fragment that binds to a CD38 expressing antibody-secreting cell and leads to the depletion of such CD38 expressing antibody-secreting cell, wherein the specific cell killing of the antibody-secreting cell is at least 10%, at least 15%, at least 20%, at least 25%, at least 30%, at least 35%, at least 40% and wherein the specific cell killing of antibody-non-secreting NK cells is less than 30%, less than 25%, less than 20%, or less than 15% as determined in a standard ADCC assay.

### WORKING EXAMPLES

### Example 1: Evaluation of Efficacy and Safety of the Human Anti-CD38 Antibody Felzartamab (MOR202) in Subjects with IgA Nephropathy (IgAN)

### 1.1 Study Design

Aim of the study is to evaluate the efficacy and safety of the human anti-CD38 antibody feczartamab (MOR202) in patients with IgA nephropathy (IgAN). Study objectives and endpoints are summarized in Table 2.

TABLE 2
Study objectives and endpoints

| Objectives | Endpoints |
|---|---|
| Primary | |
| To assess the efficacy | Relative change in UPCR in |
| of felzartamab | 24 h urine at 9 months |
| compared to placebo | compared to the reference |
| in patients with IgAN | proteinuria value in each |
| based on the change | felzartamab dose group |
| in urine protein to | vs. placebo. |
| creatinine ratio (UPCR) | |
| at 9 months. | |
| Secondary | |
| To assess the efficacy | |
| of felzartamab | |
| compared to placebo in | |
| patients with IgAN | |
| based on the following: | |
| Change in UPCR at 3, | Relative change in UPCR |
| 6, 12, 18 and 24 | in 24 h urine at 3, 6, 12, |
| months. | 18 and 24 months compared |
| | to the reference |
| | proteinuria value in each |
| | felzartamab dose group |
| | vs. placebo. |
| Complete response [CR]) | CR at 3, 6, 9, 12, 18 and |
| at 3, 6, 9, 12, 18 | 24 months in each |
| and 24 months. | felzartamab dose group |
| | vs. placebo. |
| Proportion of patients | Proportion of patients |
| with response at 3, 6, | with response at 3, 6, 9, 12, |
| 9, 12, 18 and 24 months. | 18 and 24 months in each |
| | felzartamab dose group |
| | vs. placebo. |
| Albumin-creatinine | ACR from 24 h urine at |
| ratio (ACR) at 6, 9, 12, | 6, 9, 12, 18 and 24 months |
| 18 and 24 months. | in each felzartamab dose |
| | group vs. placebo. |
| Duration of response. | Duration of response in |
| | each felzartamab dose |
| | group vs. placebo. |
| Time to response. | Time to response in each |
| | felzartamab dose group |
| | vs. placebo. |
| To assess the renal | Renal function (determine |
| function of felzartamab | by estimated glomerular |
| compared to placebo | filtration rate [eGFR] over |
| in patients with IgAN. | time) in each felzartamab |
| | dose group vs. placebo. |
| To assess the safety of | Frequency, incidence, |
| felzartamab in patients | seriousness, relatedness, and |
| with IgAN. | severity of treatment-emergent |
| | adverse events |
| | (TEAEs) across all |
| | treatment groups. |
| To assess the | Serum concentrations of |
| pharmacokinetic (PK) profile of | felzartamab over time in each |
| felzartamab in patients with IgAN. | felzartamab dose group. |
| To investigate the potential | Formation of anti-drug |
| immunogenicity of | antibodies (ADAs) over time in |
| felzartamab in patients with IgAN | all groups |
| Exploratory | |
| To assess galactose-deficient | Gd-IgA1 and anti-GD-IgA1 |
| IgA1 antibodies | antibodies serum levels over |
| (Gd-IgA1) and anti- | time in each felzartamab |
| galactose-deficient IgA1 | dose group. |
| antibodies (anti-GD-IgA1) | |
| determined by | |
| serum levels over time | |
| in patients with IgAN. | |
| To evaluate the association | Relative change over time in |
| of peripheral | peripheral immune cell |
| immune cell counts, | counts, plasma cell signature, |
| plasma cell signature, | total and antigen specific |
| total and antigen | immunoglobulin levels, |
| specific immunoglobulin | pro-inflammatory cytokine |
| levels, pro-inflammatory | levels, and complement levels |
| cytokine levels, and | with observed clinical |
| complement levels with | responses in each |
| observed clinical | felzartamab dose group. |
| responses to felzartamab | |
| over time in patients | |
| with IgAN. | |
| To evaluate the excretion | Urine levels of felzartamab |
| of felzartamab by | over time in each |
| renal filtration over time | felzartamab dose group. |
| in patients with IgAN. | |
| To evaluate the effects | Presence (yes/no) of hematuria |
| of felzartamab on | and change over time |
| hematuria in patients with IgAN. | in each felzartamab dose group. |

1.2 Study Population

### Inclusion Criteria

1. Patients of age ≥18 to ≤80 years.
2. Biopsy confirmed diagnosis of IgAN.
3. Proteinuria at screening visit ≥1.0 g/d.
4. Treatment with an angiotensin-converting enzyme inhibitor and/or angiotensin receptor blocker at maximum doses or maximally tolerated doses for 3 months and adequate blood pressure (recommended is <125 mm Hg systolic and <75 mm Hg diastolic).
5. A female is only eligible to participate if she is not pregnant, not breast feeding, and agrees to follow the contraceptive guidance during the treatment period and for at least 3 months after the last dose of felzartamab.

Kidney biopsies are performed as per institutional practice and analyzed according the MEST-C score for IgAN (see Table 3).

TABLE 3
Pathological variables used in MEST-C Score for IgAN
(Trimarchi et al. 2017).

| Variable | Definition | Score |
|---|---|---|
| Mesangial | <4 Mesangial cells/mesangial | M0 ≤ 0.5 |
| hypercellularity | area = 0 | M1 > 0.5 |
| | 4-5 Mesangial cells/mesangial | If more than half |
| | area = 1 | the glomeruli |
| | 6-7 Mesangial cells/mesangial | have more than |
| | area = 2 | three cells in a |
| | >8 Mesangial cells/mesangial | mesangial area, |
| | area = 3 | this is |
| | The mesangial hypercellularity | categorized as |
| | score is the mean | M1. Therefore, |
| | score for all glomeruli. | a formal mesangial |
| | Mesangial score | cell count |
| | should be assessed | is not always |
| | in periodic acid- | necessary to |
| | Schiff-stained sections. | derive the |
| | | mesangial score. |
| Segmental | Any amount of the | S0: absent |
| glomerulosclerosis | tuft involved in | S1: present |
| | sclerosis, but not | |
| | involving the whole | |
| | tuft or the presence | |
| | of an adhesion | |
| Endocapillary | Hypercellularity | E0-absent |
| hypercellularity | due to increased | E1-present |
| | number of cells | |
| | within glomerular | |
| | capillary lumina | |
| | causing narrowing of | |
| | the lumina | |
| Tubular atrophy/ | Percentage of cortical | T0: 0-25% |
| interstitial fibrosis | area involved by | T1: 26-50% |
| | the tubular atrophy | T2: >50% |
| | or interstitial | |
| | fibrosis, whichever | |
| | is greater | |
| Crescents | Cellular and fibrocellular | C0: no crescents |
| | crescents | C1: crescents in |
| | | <25% of glomeruli |
| | | C2: crescents in |
| | | >25% of glomeruli |

### Exclusion Criteria

Patients are excluded from the study if any of the following criteria apply:
1. Secondary forms of IgAN, indicated by the presence of any other systemic disease potentially leading to IgA deposits (e.g. Lupus nephritis, Schönlein-Henoch purpura, ankylosing spondylitis, dermatitis herpetiformis, chronic liver disease, inflammatory bowel disease, celiac disease).
2. Severe renal impairment as defined by estimated GFR <30 mL/min (using chronic kidney disease-epidemiology collaboration [CKD-EPI] formula) or the need for dialysis or renal transplant.
3. Rapidly progressive variant of IgAN, defined as eGFR loss by more than 30% per 3 months and not explained by changes in renin angiotensin system (RAS) blockade.
4. Minimal change variant of IgAN.
5. Concomitant other progressive glomerulonephritis or non-immunologic glomerular disease such as diabetic nephropathy.
6. Recipients of a kidney transplant.
7. Systemic immunosuppression (e.g. mycophenolate mofetil [MMF], cyclophosphamide, biologics like rituximab [RTX]), in particular corticosteroid therapy exceeding 20 mg/day prednisone-equivalent for more than 7 consecutive days.
8. Any previous treatment with an anti-CD38 antibody.
9. Body mass index (BMI)>35 kg/m².
10. Hemoglobin<70 g/L (4.9 mmol/L).
11. Thrombocytopenia: Platelets<100.0×10⁹/L.
12. Neutropenia: Neutrophils<1.5×10⁹/L.
13. Leukopenia: Leukocytes<3.0×10⁹/L.
14. Diabetes mellitus type 1.
15. Diabetes mellitus type 2: Patients with type 2 diabetes mellitus may only enter the clinical trial if a kidney biopsy shows IgAN without evidence of diabetic nephropathy and their disease is controlled, such as:
   a. Glycated hemoglobin (HbAlc)<8.0% or <64 mmoL/mol.
   b. No diabetic retinopathy known.
   c. No peripheral neuropathy known.
16. Significant uncontrolled cardiovascular disease (including arterial or venous thrombotic or embolic events) or cardiac insufficiency (New York Heart Association [NYHA] class IV).
17. Clinically relevant findings on a 12-lead electrocardiogram (ECG) as determined by the investigator at screening.
18. History of significant cerebrovascular disease or sensory or motor neuropathy of toxicity≥grade 3.
19. Aspartate aminotransferase or alanine aminotransferase>1.5×ULN, alkaline phosphatase>3.0×ULN.
20. Known or suspected hypersensitivity to felzartamab and its excipients (L-histidine, sucrose, polysorbate 20).
21. Serologic or virologic markers positive for HIV, hepatitis C (patients with positive anti-hepatitis C virus [anti-HCV] antibody but negative HCV RNA-PCR can enroll) or active or latent hepatitis B (patients with positive hepatitis B surface antigen [HBsAg] are excluded). For patients with isolated positive hepatitis B core antibody [anti-HBc], hepatitis B virus (HBV) DNA test by PCR must be non-detectable to enroll).
22. Any malignancy within 5 years prior to screening start, with the exception of adequately treated in situ carcinoma of the cervix uteri, basal or squamous cell carcinoma or other non-melanomatous skin cancer.
23. Any active infection (viral, fungal, bacterial) requiring systemic therapy.

1.3 Dosing

Patients will receive nine infusions of either felzartamab or placebo on Day 1, 8, 15, 22, 29, 57, 85, 113 and 141 according to Table 4.

TABLE 4
Dosing arms

| Dosing | | |
|---|---|---|
| arm | felzartamab (MOR202) | placebo |
| M1 | 2 Doses: | Day 8, 22, 29, 57, 85, |
| | Day 1 and 15 | 113 and141 |
| M2 | 5 Doses: | Day 22, 85, 113 and141 |
| | Day 1, 8, 15, 29 and 57 | |
| M3 | 9 Doses: | none |
| | Day 1, 8, 15, 22, 29, 57, 85, | |
| | 113 and 141 | |
| Control | none | Day 1, 8, 15, 22, 29, |
| arm | | 57, 85, 113 and 141 |

Felzartamab will be dosed depending on patient body weight (Table 5). The absolute dose to be administered intravenously (i.v.) will be determined according to the following information:

TABLE 5
Felzartamab (MOR202) dose by body weight

| Body weight [kg] | ≤50 | >50 to 70 | >70 to 90 | >90 |
|---|---|---|---|---|
| felzartamab dose [mg] | 650 | 975 | 1300 | 1625 |
| Number of felzartamab vials | 2 | 3 | 4 | 5 |

Each patient will receive 650 mg to 1625 mg felzartamab per dose i.v. depending onthi individual body weight. Dosing in this trial is based on the results of the FIH trial in MM (MOR202C101) as well as a PK/PD modeling approach (Raab M S et al. (2020) Lancet Haematol.7(5):e381-e394 2020). Within 4 specified body weight ranges, a fixed dosing concept will be employed to simplify the dosing procedure. The 4 dose levels for the 4 body weight ranges were chosen to be similar to a 16 mg/kg dose (i.e. the recommended dose in the FIH study MOR202C101) as shown in Table 6.

TABLE 6
Felzartamab fixed dosing in comparison to body weight dosing

| Body weight [kg] | 40 | 50 | 50.5 | 60 | 70 | 70.5 | 80 | 90 | 90.5 | 100 | 130 |
|---|---|---|---|---|---|---|---|---|---|---|---|
| Fixed dose used | 650 | | 975 | | | 1300 | | | 1625 | | |
| in current study | | | | | | | | | | | |
| [mg] | | | | | | | | | | | |
| Number of | 2 | | 3 | | | 4 | | | 5 | | |
| Felzartamab vials | | | | | | | | | | | |
| (325 mg per vial) | | | | | | | | | | | |
| Corresponding | 16.3 | 13.0 | 19.3 | 16.3 | 13.9 | 18.4 | 16.3 | 14.4 | 18.0 | 16.3 | 12.5 |
| body weight | | | | | | | | | | | |
| dosing [mg/kg] | | | | | | | | | | | |

The patients will be infused with felzartamab in 0.9% saline or placebo (0.9% saline only). Premedication to reduce the risk of IRRs is administered 2 hours to 30 minutes prior to each infusion:
Oral paracetamol (acetaminophene) 650 to 1000 mg.
Oral or i.v. diphenhydramine 25 to 50 mg or equivalent drug and dose.
i.v. corticosteroid according to Table 7.

If no IRRs occur, infusion speed may be increased and glucocorticoid premedication may be reduced according to Table 7. For patients who do not experience ≥Grade 2 IRRs/≥Grade 1 cytokine release syndrome to felzartamab/placebo during the first three cycles, premedication will be optional for subsequent infusions. Otherwise, the premedication should be continued for subsequent administrations.

TABLE 7
Felzartamab/placebo infusion speed and i.v. corticoid premedication

| Felzartamab/placebo | | | | 4th and |
|---|---|---|---|---|
| infusion number | 1 | 2 | 3 | onwards |
| Recommended | 2 mL/min | 4 mL/min | 8 mL/min | 8 mL/min |
| maximum | | | | |
| infusion speed | | | | |
| methylprednisolone | 100 mg | 100 mg | 50 mg | not |
| or equivalent | | | | mandatory |

1.4 Efficacy Assessment

Planned time points for all efficacy assessments are provided in Example 1.1 and efficacy objectives and endpoints are shown in Table 2.

Efficacy parameters are defined in Table 8.

TABLE 8
Efficacy parameters

| Efficacy | |
|---|---|
| parameter | Definition |
| Proteinuria | Proteinuria changes are reflected by the |
| assessment | reduction of proteinuria as measured |
| | by UPCR. They will evaluated as a |
| | continuous variable. The reference |
| | proteinuria value before start of treatment |
| | is defined as the mean of the values |
| | determined at screening and prior to |
| | baseline (visit 2) predose (UPCR from |
| | 24 h urine). |
| Complete | Reduction of proteinuria to less than |
| Response | 0.3 g/g UPCR, serum albumin within the |
| (CR) | reference range of the central laboratory |
| | and stable eGFR (at least 80% of |
| | value at baseline visit) |
| Response | Reduction of proteinuria to below |
| | 0.6 g/g (UPCR) and stable eGFR (at least |
| | 80% of value at baseline visit), but not CR. |
| Duration of | Date of 1st observation of progressive |
| response | disease minus date of 1st observation |
| | of response + 1 day |
| Progressive | Decrease of eGFR by more than 30% |
| disease | of baseline eGFR, or increase in urine |
| | protein: creatinine ratio (UPCR) by more |
| | than 50% from baseline value in non- |
| | responding patients or more than |
| | 25% over nadir in responding patients |
| Time to CR | Determined as date of 1st observation |
| | of CR minus date of randomization + 1 |
| | day |
| Time to | Determined as date of 1st observation |
| Response | of Response minus date of |
| | randomization + 1 day |
| Estimated | eGFR is be calculated as per the chronic kidney |
| glomerular | disease epidemiology collaboration (CKD-EPI) |
| filtration rate | equation (Levey et al 2007, Levey et al |
| (eGFR) | 2009) |

Proteinuria and UPCR will be determined from 24-hour urine samples. If the collected urine does not contain at least 5 mg creatinine/kg/day for females and 6 mg creatinine/kg/day for males, urine collection needs to be repeated immediately without undue delay.

### Example 2: Evaluation of Efficacy and Safety of the Human Anti-CD38 Antibody Felzartamab (MOR202) in Subjects with Lupus Nephritis (LN)

### 2.1 Study Design

Aim of the study is to evaluate the efficacy and safety of the human anti-CD38 antibody felzartamab (MOR202) in patients with Lupus Nephritis (LN). Patients will be randomized to receive one of 3 different dosing schedules of felzartamab (dosing arms M1, M2 or M3) or placebo. All patients will receive background LN therapy with mycophenolate mofetil (MMF)/mycophenolic acid (MPA) and hydroxychloroquine (if not contraindicated and available) as well as angiotensin-converting enzyme inhibitor (ACEi) and/or angiotensin receptor blocker (ARE) throughout the trial. Corticosteroids will be tapered down to a minimum or removed if the patient is doing well.

TABLE 9
Study objectives and endpoints

| Objectives | Endpoints |
|---|---|
| Primary | |
| To assess the efficacy of 3 different dosing | Relative change in UPCR |
| regimen of felzartamab in comparison to | in 24 h urine at 12 months |
| placebo in addition to background treatment | compared to baseline. |
| (MMF or MFA) in patients with LN. | |

Secondary
To assess the efficacy of 3 different dosing regimen
of felzartamab vs. placebo in terms of the following:
overall response in LN (CR or PR) at 3, 6, 9, and 12 months
complete response in LN at 3, 6, 9, and 12 months
partial response in LN at 3, 6, 9, and 12 months
of time to response.
duration of response.
To assess the safety of Felzartamab determined by
frequency, incidence, seriousness, relatedness, and
severity of treatment-emergent adverse events (TEAEs)
To assess the PK profile of Felzartamab
determined by serum concentrations over time.
To investigate the potential immunogenicity of
felzartamab determined by formation of anti-drug
antibodies.
To assess the renal function determined by eGFR over time.
Exploratory
To evaluate the association of peripheral immune cell
counts, total and antigen specific immunoglobulin
levels, pro-inflammatory cytokine levels, and
complement levels (including C3) determined by change
from baseline over time
To evaluate the excretion of Felzartamab by renal
filtration determined by urine levels over time
To assess anti-double-stranded DNA antibodies
(dsDNA) determined by serum levels over time
To assess the efficacy of 3 different dosing
regimens of felzartamab vs. placebo in terms of relative
change of albumin-creatinine ratio (ACR)
from 24 h urine at 3, 6, 9 and 12 months
To evaluate changes in SLE Disease Activity Index
(SLEDAI) 2K, Systemic Lupus International
Collaborating Clinics/American College of
Rheumatology damage index (SLICC/ACR DI) from baseline
over time.
To evaluate changes in EQD5L5 and in fatigue
assessment score from baseline over time.

### 2.2 Study Population

### Inclusion Criteria

1. Patients of age ≥18 and ≤80 years
2. Classification as SLE, according to current EULAR/ACR 2019 criteria.
3. Class III or IV LN as evidenced by renal biopsy performed within 12 months prior to or during screening according to International Society of Nephrology/Renal Pathology Society 2003 classification. Patients may co-exhibit Class V disease in addition to either Class III or Class IV disease
4. Proteinuria at screening visit ≥0.75 g/24 h.
5. Treatment with an angiotensin-converting enzyme inhibitor (ACEi) and/or angiotensin receptor blocker (ARB) at maximum doses or maximally tolerated doses for ≥3 months and adequate blood pressure <130 mm Hg systolic and <80 mm Hg diastolic
6. Estimated glomerular filtration rate (eGFR)≥30 mL/min/1.73 m².

### Exclusion Criteria

Patients will be excluded from the trial if any of the following criteria apply:
1. Presence of rapidly progressive glomerulonephritis defined by (a) presence of crescent formation in ≥50% of glomeruli assessed on renal biopsy or (b) sustained doubling of serum creatinine within 12 weeks of screening or (c) the investigator's opinion that the patient has rapidly progressive glomerulonephritis.
2. Recipients of a kidney transplant.
3. Greater than 50% of glomeruli with sclerosis on renal biopsy and interstitial fibrosis and tubular atrophy score (IFTA)<65%.
4. Prior to screening start, oral or parenteral treatment with: (a) Alkylating agents (e.g. cyclophosphamide) or calcineurin inhibitors (CNIs) (e.g. tacrolimus, cyclosporine A) within 90 days prior to signing of ICF or (b) biologic drugs including rituximab (RTX) within 180 days or (c) any other oral/parenteral IST except MMF/MPA or hydroxychloroquine (or other chloroquine compound) within 180 days or (d) induction therapy with MMF/MPA plus corticosteroids started more than 42 days prior to signing ICF.
5. Any previous treatment with an anti-CD38 antibody.
6. Hemoglobin<70 g/L, unless caused by autoimmune hemolytic anemia resulting from SLE.
7. Thrombocytopenia: Platelets<50.0×10⁹/L.
8. Unstable disease with thrombocytopenia or at high risk for developing clinically significant bleeding or organ dysfunction requiring therapies such as plasmapheresis or acute blood or platelet transfusions.
9. Neutropenia: Neutrophils<1.5×10⁹/L.
10. Leukopenia: Leukocytes<2.5×10⁹/L.
11. B-cells<5×10⁶/L.
12. Diabetes mellitus type 1 or 2.
13. Significant uncontrolled cardiovascular disease (incl. arterial or venous thrombotic or embolic events) or cardiac insufficiency (New York Heart Association [NYHA] class IV) as judged by the investigator.
14. Clinically relevant findings on a 12-lead electrocardiogram (ECG) as determined by the investigator at screening.
15. History of significant cerebrovascular disease or sensory or motor neuropathy of toxicity ≥grade 3.
16. Aspartate aminotransferase or alanine aminotransferase>1.5×ULN, alkaline phosphatase>3.0×ULN.
17. Known or suspected hypersensitivity to felzartamab and its excipients (L-histidine, sucrose, polysorbate 20).
18. Intolerance or contraindication to systemic corticosteroids, MMF or MPA
19. Serologic or virologic markers positive for HIV, hepatitis C (patients with positive anti hepatitis C virus [anti-HCV] antibody but negative HCV RNA polymerase chain reaction can enroll) or active or latent hepatitis B (patients with positive hepatitis B surface antigen [HBsAg] are excluded). For patients with isolated positive hepatitis B core antibody [anti-HBc], hepatitis B virus (HBV) DNA test by PCR must be non-detectable to enroll).
20. For any other preexisting symptoms and impairments of health or any residual toxicity from prior therapy classified ≥grade 3 (NCI-CTCAE, see Appendix 4): these patients may be included upon confirmation by the Medical Monitor.
21. Any malignancy within 5 years prior to screening start, with the exception of adequately treated in situ carcinoma of the cervix uteri, basal or squamous cell carcinoma or other non-melanomatous skin cancer.
23. Any active infection (viral, fungal, bacterial) requiring systemic therapy.
24. Significant or uncontrolled medical disease in any organ system not related to SLE or LN, which, in the investigator's opinion, would preclude patient participation.
25. Retinitis, poorly controlled seizure disorder, acute confusional state, myelitis, stroke or stroke syndrome, cerebellar ataxia, or dementia that is currently active and resulting from SLE.

2.3 Dosing

Patients will receive nine infusions of either felzartamab or placebo on day 1, 8, 15, 22, 29, 57, 85, 113 and 141 according to Table 4.

Felzartamab will be dosed depending on patient body weight. The absolute dose to be administered intravenously (i.v.) will be determined according to Table 5. Each patient will receive 650 mg to 1625 mg Felzartamab per dose i.v. depending on their individual body weight. Dosing in this trial is based on the results of the FIH trial in MM (MOR202C101) and a PK/PD modeling approach (Raab M S et al. (2020) Lancet Haematol.7(5):e381-e394 2020). Within 4 specified body weight ranges, a fixed dosing concept will be employed to simplify the dosing procedure. The 4 dose levels for the 4 body weight ranges were chosen to be similar to a 16 mg/kg dose (i.e. the recommended dose in the FIH study MOR202C101) as shown in Table 6. The patients will be treated with the investigational medicinal product (IMP) Felzartamab/Placebo (IMP) and LN background therapy. IMP should be administered in an environment where resuscitation is possible. The following medications to reduce the risk of infusion-related reactions (IRRs) should be administered 60 to 30 minutes prior to each infusion:
Oral paracetamol (acetaminophene) 650 to 1000 mg.
Oral or i.v. diphenhydramine 25 to 50 mg or equivalent drug and dose.
i.v. glucocorticoid according to Table 7

If no IRRs occur, infusion speed may be increased and glucocorticoid premedication may be reduced according to Table 7.

### 2.4 Efficacy Assessment

Planned time points for efficacy assessments are provided in Example 2.1 and efficacy objectives and endpoints are shown in Table 9. Efficacy parameters are as defined in Table 8.

### Example 3: In Vitro and In Vivo Evaluation of Felzartamab (MOR202) Using Samples from IgA Nephropathy (IgAN) and Lupus Nephritis (LN) Patients

### 3.1 In Vitro Studies

Aim of the in vitro studies is to evaluate the ability of the human anti-CD38 antibody felzartamab (MOR202) to deplete CD38+ long-lived plasma cells. Plasma cells are very likely the main cell type secreting autoantibodies against self-antigens in IgAN and LN, leading to the formation of ICs and glomerular inflammation. As plasma cells are a very rare cell population in the peripheral blood of healthy as well as patients with IgAN and LN, the setup of an in vitro assay using plasma cells directly from peripheral blood has proven not feasible. Thus, in a first step, an in vitro differentiation assay as described by Cocco M et al. ((2012) J Immunol 189(12):5773-85) is used to differentiate the more abundantly present memory B cells (Bmem) into long-lived plasma cells. In brief, peripheral blood mononuclear cells (PBMCs) are isolated from whole blood by density gradient centrifugation, and subsequently, Bmem are then isolated by magnetic cell sorting from the PBMCs. Bmem are cultured in the presence of varying cytokine cocktails and supporting feeder cell lines for a period of at least 16 days until fully differentiated as confirmed by flow cytometric staining.

Secretion of human IgG by the differentiated plasma cells is confirmed by ELISA. Further ELISAs are performed to detect anti-GD-IgA1 and anti-nuclear antibodies and to assess the levels of these disease-specific autoantibodies secreted from plasma cells differentiated from IgAN and LN patient B cell samples, respectively.

The in vitro-differentiated patient-derived plasma cells are also tested in ADCC assays, where the plasma cells as target cells are co-cultured with NK cells as effector cells, and incubated together either with felzartamab or an isotype control antibody. The percentage of depletion of CD38+ plasma cells by felzartamab vs. control is evaluated by flow cytometry. Additionally, in vitro-differentiated patient-derived plasma cells are co-cultured with NK cells and felzartamab or isotype control antibody as described above, and ELISAs measuring total human IgG as well as disease-specific autoantibody levels in the cell culture supernatant are performed to demonstrate that total and disease-specific antibody levels are drastically reduced since antibody-secreting plasma cells have been depleted in the culture after felzartamab treatment. Additionally, a shortened differentiation protocol (adapted from Wang T et al., (2019) Front Immunol. 10:1243) is used to confirm the ability of felzartamab to deplete CD38+ cells in vitro in a different setting. Employing this assay, patient PBMCs are co-cultured with a TLR7/8 agonist, IL-2, and IFNα2b, as well as either felzartamab or an isotype control antibody. After a cultivation period of 5-6 days, depletion of CD38+CD27+ cells by felzartamab is confirmed by flow cytometry and decrease of IgG secretion as compared to cells treated with the isotype control antibody by ELISA.

### 3.2 In Vivo Studies

The in vivo studies aim to demonstrate the potential of felzartamab to deplete autoantibody secreting plasma cells, thus reducing autoantibody titers and intensity of autoimmunity symptoms in vivo, in a disease relevant mouse model.

Since the CD38 biology in rodents is very different when compared to human, the studies involve immunocompromised mice engrafted with human immune components.

The lupus nephritis model is based on the well-established pristane-induced autoimmunity model, but in immunocompromised NSG mice engrafted with human CD34+ cells from cord blood (Gunawan M et al. (2017) Sci Rep, 7(1):16642). The mice develop human SLE-like symptoms (human autoantibody (nuclear antibodies) production, lupus nephritis and pulmonary serositis, lymphopenia). The readout includes reduction of plasma cells in peripheral blood, spleen and bone marrow (evaluated by flow cytometry), as well as reduction of pathogenic anti-nuclear antibodies in the serum (measured by ELISA).

Since it is very difficult to model IgA nephropathy in a humanized setting, the only feasible option is to demonstrate the potential of felzartamab to deplete human plasma cells producing antibodies against galactose-deficient IgA (gdlgA) antibodies in vivo. The model is based on a gdlgA vaccination in NSG-SGM3 mice, humanized with CD34+ cells. It has been reported that human CD38+ plasma cells can be present in this mouse strain (Jangalwe S et al. (2016) Immun Inflamm Dis 4(4):427-440). The readout includes reduction of plasma cells in peripheral blood, spleen and bone marrow (evaluated by flow cytometry), as well as reduction of anti-gdlgA antibodies in the serum (measured by ELISA).

1. An anti-CD38 antibody or antibody fragment for use in the treatment of an immune complex-mediated disease in a subject.

2. An anti-CD38 antibody or antibody fragment for use according to claim 1, wherein the immune complex—mediated disease is a kidney disease.

3. An anti-CD38 antibody or antibody fragment for use according to claim 2, wherein the immune complex—mediated disease is selected from IgA nephropathy, lupus nephritis, Henoch-Schönlein purpura nephritis, post-streptococcal glomerulonephritis or drug-induced immune complex—mediated diffuse proliferative glomerulonephritis.

4. An anti-CD38 antibody or antibody fragment for use according to claim 3, wherein the IgA nephropathy is a galactose-deficient IgA1 antibody (Gd-IgA1) and anti-galactose-deficient IgA1 antibody (anti-GD-IgA1) positive IgA nephropathy.

5. An anti-CD38 antibody or antibody fragment for use according to any of the preceding claims, wherein the antibody comprises a HCDR1 region of amino acid sequence SEQ ID NO.: 1, a HCDR2 region of amino acid sequence SEQ ID NO.: 2, a HCDR3 region of amino acid sequence SEQ ID NO.: 3, and a LCDR1 region of amino acid sequence SEQ ID NO.: 4, a LCDR2 region of amino acid sequence SEQ ID NO.: 5 and a LCDR3 region of amino acid sequence SEQ ID NO.: 6.

6. An anti-CD38 antibody or antibody fragment for use according to claim 5, wherein anti-CD38 said antibody or antibody fragment comprises a variable heavy chain (VH) region of SEQ ID NO.: 7 and a variable light chain (VL) region of SEQ ID NO.: 8.

7. An anti-CD38 antibody or antibody fragment for use according to any of the preceding claims, wherein said antibody or antibody fragment specific for CD38 is an IgG1.

8. An anti-CD38 antibody or antibody fragment for use according to any of the preceding claims, wherein said antibody or antibody fragment specific for CD38 is a human antibody.

9. An anti-CD38 antibody or antibody fragment for use according to any of the preceding claims, wherein the antibody depletes plasma cells by ADCC and/or ADCP.

10. An anti-CD38 antibody or antibody fragment for use according to any of the preceding claims, wherein administration of said anti-CD38 antibody or antibody fragment leads to a reduction of immune complexes.

11. An anti-CD38 antibody or antibody fragment for use according to any one of claims 4 to 10, wherein the immune complex comprises Gd-IgA1 and anti-GD-IgA1 antibodies.

12. An -CD38 antibody or antibody fragment for use according to claim 10 or 11, wherein the immune complex is deposited in the glomeruli of the kidney.

13. The anti-CD38 antibody or antibody fragment for use according to any of the preceding claims, wherein the antibody or antibody fragment will be dosed depending on subject body weight.

14. The anti-CD38 antibody or antibody fragment for use according to claim 13, wherein the antibody or antibody fragment will be dosed in at least 2 doses, at least 5 doses, or at least 9 doses according to Table 6.

15. The anti-CD38 antibody or antibody fragment for use according to any of the preceding claims, wherein the subject to be treated is characterized by proteinuria at screening of ≥1.0 g/day.


SEQUENCE LISTING (11 sequences)

SEQ ID NO: 1 - protein, 5 aa, Artificial Sequence
  source: /note=“Description of Artificial Sequence Synthetic peptide”
SYYMN

SEQ ID NO: 2 - protein, 17 aa, Artificial Sequence
  source: /note=“Description of Artificial Sequence Synthetic peptide”
GISGDPSNTYYADSVKG

SEQ ID NO: 3 - protein, 11 aa, Artificial Sequence
  source: /note=“Description of Artificial Sequence Synthetic peptide”
DLPLVYTGFAY

SEQ ID NO: 4 - protein, 11 aa, Artificial Sequence
  source: /note=“Description of Artificial Sequence Synthetic peptide”
SGDNLRHYYVY

SEQ ID NO: 5 - protein, 7 aa, Artificial Sequence
  source: /note=“Description of Artificial Sequence Synthetic peptide”
GDSKRPS

SEQ ID NO: 6 - protein, 9 aa, Artificial Sequence
  source: /note=“Description of Artificial Sequence Synthetic peptide”
QTYTGGASL

SEQ ID NO: 7 - protein, 120 aa, Artificial Sequence
  source: /note=“Description of Artificial Sequence Synthetic polypeptide”
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 8 - protein, 109 aa, Artificial Sequence
  source: /note=“Description of Artificial Sequence Synthetic polypeptide”
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 9 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 10 - DNA, 360 bp, Artificial Sequence
  source: /note=“Description of Artificial Sequence Synthetic polynucleotide”
caggtgcaattggtggaaagcggcggcggcctggtgcaaccgggcggcagcctgcgtctgagctgcgcggcctccggatttaccttttcttcttattatatgaattgggtgcgccaagcccctgggaagggtctcgagtgggtgagcggtatctctggtgatcctagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatcttcctcttgtttatactggttttgcttattggggccaaggcaccctggtgacggttagctca

SEQ ID NO: 11 - DNA, 327 bp, Artificial Sequence
  source: /note=“Description of Artificial Sequence Synthetic polynucleotide”
gatatcgaactgacccagccgccttcagtgagcgttgcaccaggtcagaccgcgcgtatctcgtgtagcggcgataatcttcgtcattattatgtttattggtaccagcagaaacccgggcaggcgccagttcttgtgatttatggtgattctaagcgtccctcaggcatcccggaacgctttagcggatccaacagcggcaacaccgcgaccctgaccattagcggcactcaggcggaagacgaagcggattattattgccagacttatactggtggtgcttctcttgtgtttggcggcggcacgaagttaaccgttcttggccag